(19) United States
(12) Patent Application Publication
(10) Pub. No.: US 2005/0084513 A1
(43) Pub. Date: Apr. 21, 2005

(54) Nanocoating for improving biocompatibility of medical implants

(75) Inventor: Liping Tang, Arlington, TX (US)

Correspondence Address: KENNETH R. GLASER;MONIQUE A. VANDER MOLEN, GARDERE WYNNE SEWELL LLP, 1601 ELM STREET, SUITE 3000, DALLAS, TX, 75201-4761, US

(73) Assignee: Board of Regents, Austin, TX (US)

(21) Appl. No.: 10/896,376

(22) Filed: Jul. 21, 2004

Related U.S. Application Data
(63) Continuation-in-part of application No. 10/690,466, filed on Oct. 21, 2003.

(51) Int. Cl. A61F 2/00; A61K 9/14

(52) U.S. Cl. 424/423; 424/489

(57) ABSTRACT

A coating for an implant surface comprising one or more nanoparticles of less than or equal to 500 nanometers and an implant surface capable of receiving the nanoparticles, the implant selected from the group consisting of metal, carbon, graphite, polymer, protein, nucleic acid, microorganisms, hydrogel, liquid, porous and polymer blend particles, and combinations thereof. The coating promotes characteristics on the implant surface such as reducing protein unfolding, preventing inflammatory and fibrotic cell accumulation, reducing the number of such cell attachment sites and preventing other adverse biological reactions. The coating may be applied on any material via physical and/or chemical binding. The coating may further comprise a surfactant and may include a tag, adsorbed, absorbed or incorporated onto the nanoparticle. The coating on an implant surface is used for purposes that may be cosmetic, therapeutic, preventative, reconstructive, monitoring and replacement. The coating may also be used for in vitro purposes.

### CROSS-REFERENCES TO RELATED APPLICATIONS

This application is a continuation-in-part of U.S. patent application Ser. No. 10/690,466, filed Oct. 21, 2003, incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of EB-00287 awarded by The National Institutes of Health.

### BACKGROUND OF THE INVENTION

The present invention relates generally to the field of medical implants and in particular to providing medical implants with improved biocompatibility.

Medical implants and devices play an important role in the practice of contemporary medicine. Unfortunately, following introduction into an organism, many implants and devices trigger a series of biologic reactions, many of which are deleterious to the body. Such adverse biologic reactions include inflammation, fibrosis, thrombosis, and infections that may lead to implant rejection.

One component leading to these adverse reactions is implant-mediated protein “denaturation,” a biologic process that appears to occur via protein adsorption onto the surface of an implant. The adsorption is led by a chaotic layer of spontaneously adsorbed, partially ‘denatured’ host proteins, including fibrinogen. (Tang L and Eaton J W, Fibrin(ogen) mediates acute inflammatory responses to biomaterials. J Exp Med 1993;178:2147-56; Hu et al. Molecular basis of biomaterial-mediated foreign body reactions. Blood 2001;98:1231-38; incorporated herein by reference). The denatured proteins, such as fibrinogen, are thus involved in promoting adverse biologic reactions to an implant, by, in part, attracting inflammatory cells to implants after their adsorption. Unfortunately, it remains to be understood how to prevent the denaturation and adsorption processes. Indeed, there remains a need for implants and devices that do not promote such adverse biologic reactions. This is likely to occur by identifying implants and surfaces that are compatible with the body (e.g., biocompatible) and do not promote protein denaturation and/or protein adsorption onto the implant surface.

To date, the production of biocompatible implants and devices has yielded materials with hydrophilic surfaces thought to prevent protein (e.g., fibrinogen) denaturation. Disappointingly, even the most hydrophilic of these materials, including polyethylene glycol, when placed on the surface of an implant or device is found to prompt protein conformational changes and adverse biologic reactions.

Presently, most if not all medical implants when introduced into an organism trigger a series of biologic reactions, referred to herein as foreign body reactions. The biologic reactions are generally accompanied by an accumulation of inflammatory and fibrotic cells that collect and/or adhere to the implant surface. It is this accumulation of cells, their by-products and the associated immune responses that lead to the failure of medical implants or devices.

Prior art coating techniques have been developed to improve the biocompatibility of the implant. These techniques, however, have been designed to change material surface chemistries in an attempt to reduce protein denaturation and protein/cell accumulation. Prior art techniques generally fail to significantly reduce surface-induced protein denaturation and subsequent adverse reactions. Therefore, there still remains a need for improved implants with surfaces that prevent protein denaturation and subsequent adverse reactions in the organism.

### SUMMARY OF THE INVENTION

The present invention solves many problems associated with adverse reactions occurring upon introduction of an implant or device into an organism. The present invention provides for a preparation that prevents protein denaturation (e.g., unfolding) and subsequent adverse reactions upon its introduction into an organism.

Generally, and in one form the present invention is a nanoparticle preparation that reduces or prevents protein unfolding as well as subsequence adverse reactions from occurring in an organism. Adverse reactions may include biologic processes and/or cell surface interactions such as inflammatory cell accumulation, protein unfolding, protein denaturation, fibrotic tissue formation, thrombosis and device-centered infection. The nanoparticle preparation comprises nanoparticles less than or equal to 500 nanometer (nm) in diameter and an implant surface capable of receiving the nanoparticles. As such, the invention provides for a biocompatible coating on an implant that prevents adverse reactions in the body upon its introduction into an organism.

In another form, the present invention is a nanoparticle preparation for coating an implant surface comprising nanoparticles of less than or equal to 500 nanometers, wherein the nanoparticles promote characteristics on the implant surface after implantation into an organism in need thereof, the characteristics selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof.

In yet another form, the present invention is a nanoparticle preparation for coating an implant surface comprising one or more nanoparticles of less than or equal to 500 nanometers and coating the surface of an implant with nanoparticles, wherein the nanoparticles promote characteristics on the implant surface selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof. The method may include coating an implant or device with such a nanoparticle preparation that prevents protein unfolding or denaturation upon introduction of the implant into an organism.

Advantages of the present invention include findings that the reduction or prevention of protein unfolding, adverse biologic reactions, protein adsorption and protein denaturation that occur via the present invention appear regardless or independent of nanoparticle composition. In addition, the nanoparticle preparation of the present invention does not adversely affect surface properties or function of an implant.

Those skilled in the art will further appreciate the above-noted features and advantages of the invention together with other important aspects thereof upon reading the detailed description that follows in conjunction with the drawings.

### BRIEF DESCRIPTION OF SEVERAL VIEWS OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 depicts a schematic of a nanoparticle in accordance with one aspect of the present invention;

FIG. 2A depicts a lack of foreign body reactions in mice following contact with 100 nm NIPA particles of the present invention;

FIG. 2B illustrates one example of inflammatory and fibrotic reactions in mice following contact with 10 micrometer NIPA particles;

FIG. 2C illustrates a lack of foreign body reactions in hypofibrinogenic mice following contact with microparticles of the present invention;

FIG. 2D illustrates “normal” foreign body reactions in hyperfibrinogenemic mice following contact with 10 micrometer microparticles preincubated with fibrinogen;

FIG. 2E illustrates the extent of foreign body reactions (as number of cells associated with a particle implants) in mice following contact with various coated and uncoated implants;

FIG. 3 shows fibrinogen accumulation in untreated Balb/C mice following subcutaneous implantation of (FIG. 3A) 10 micrometer microparticles or (FIG. 3C) 100 nm nanoparticles as it compares with ancrod-treated Balb/C mice following subcutaneous implantation of (FIG. 3B) 10 micrometer microparticles or (FIG. 3D) 100 nm nanoparticles;

FIG. 4 exemplifies an inflammatory response following implantation of 10 micrometer NIPA particles for views of (FIG. 4A) X200 and (FIG. 4B) X600 as it compares with the absence of such a response following implantation of 100 nm NIPA nanoparticles for views of (FIG. 4C) X200 and (FIG. 4D) X600;

FIG. 5A shows an absence of an adverse or foreign body reaction seven days after implantation of poly-L-lactic acid fibers covalently coated with 100 nm nanoparticles of the present invention;

FIG. 5B depicts an adverse or foreign body reaction seven days after implantation of “uncoated” poly-L-lactic fibers;

FIG. 6 depicts fibrinogen P2 epitope exposure on fibrinogen adsorbed to (FIG. 6A) 10 micrometer microparticles preincubated with human fibrinogen as it compares with (FIG. 6B) 100 nanometer nanoparticles preincubated with human fibrinogen, (FIG. 6C) fibrinogen-free 10 micrometer microparticles (FIG. 6D) and fibrinogen-free 100 nanometer nanoparticles; and

FIG. 7 depicts a schematic of potential nanoparticle coatings.

### DETAILED DESCRIPTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

In the description which follows, like parts may be marked throughout the specification and drawing with the same reference numerals, respectively. The drawing figures are not necessarily to scale and certain features may be shown exaggerated in scale or in somewhat generalized or schematic form in the interest of clarity and conciseness.

The present invention provides for a surface on an implant, similar to a surface “coating,” that reduces and/or prevents adverse foreign body reactions, such as protein adsorption to the implant surface. The present invention improves the biocompatibility and blood compatibility of an implant by using a coating of nanoparticles, wherein each particle is generally less than 500 nm in diameter. Thus, nanoparticles of the present invention reduce protein “denaturation” as well as subsequent foreign body reactions.

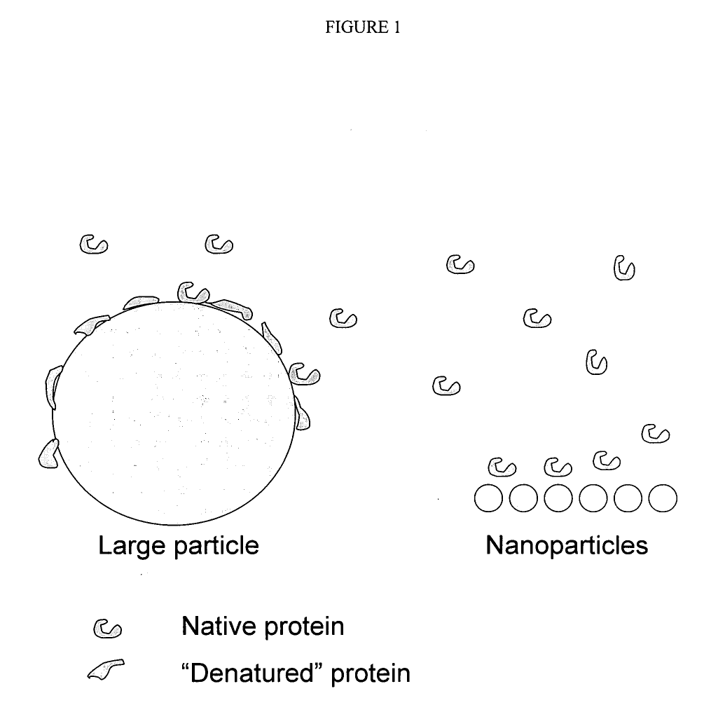

When a protein undergoes “denaturation,” or unfolds, the protein adsorbs and interacts or attaches to multiple sites on the surface of the material. By “coating” a material with particles, the number of interactions or attachment sites or the extent of protein-surface interactions are reduced. (See FIG. 1). If the particles are too large, however, the protein is still able to unfold or denature and, thus, adsorb to the particle. Thus, if the particle size and consequently the relative surface of the material is reduced to that of the size of a nanoparticle of the present invention, proteins can no longer unfold or denature. The presence of nanoparticles to an implant surface, thus, reduces the denaturation and adsorption process of proteins to the implant surface and also reduces subsequent adverse or foreign body reactions. As such, nanoparticle coating of implants, in accordance with the present invention, provides for improved biocompatibility and, subsequently, therapeutic efficacy of the implant and hence with an organism in need of such an implant.

The above improvements are independent of nanoparticle composition. Thus compositions nanoparticle preparations comprising one or more degradable polymers, nondegradable polymers, metals, proteins, nucleic acids, micro-organisms (bacteria and viruses) and similar combinations may be used to improve the biocompatibility of implants introduced to organisms.

As used herein, medical implants or devices include any material with a surface to which a “coating” may be applied. This includes implants introduced for cosmetic, reconstructive, monitoring or replacement purposes, such as a joint implant, breast implant, dental implant, chip or ion implant, brain implant, retinal implant, cochlear implant, facial implant, organ implant, and prosthesis, as examples. It also includes particles, catheters and other devices introduced into an organism, such as drug release particles, miniature sensors and stents, as examples. The implant “material” as used herein may be any organic or inorganic used with medical implants or devices.

As used herein, the “coating” applied to the material surface includes “nanoparticles,” “nanoparticles-like objects,” “microscopic particles” or “functionalized particles.” Alternatively, the material surface may be treated to create particle-like structures on the surface by performing surface modification procedures, such as plasma polymerization, spot coating, etc. Such particles are generally a few micrometers in size to few millimeters in size or submicroscopic (less than one micrometer) and solid colloidal objects that may be cylindrical or spherical in shape with a semipermeable shell or shaped like a permeable nano-ball. One or more drugs or other relevant materials, referred to as a “tag,” (e.g., used for labeling, as a molecular ligand, for diagnosis or therapy, such as for a medical treatment, nuclear medicine or radiation therapy) may be included with the nanoparticles of the present invention. Inclusion may be via entrapment, encapsulation, absorption, adsorption, covalent linkage, or other attachment. Nanoparticles of the present invention may be, themselves, further coated as required.

Nanoparticles of the present invention are generally provided as a metal particle, carbon particle, inorganic chemical particle, organic chemical particle, ceramic particle, graphite particle, polymer particle, protein particle, peptide particle, DNA particle, RNA particle, bacteria/virus particle, hydrogel particle, liquid particle or porous particle. Thus, the nanoparticles may be, for example, metal, carbon, graphite, polymer, protein, peptide, DNA/RNA, microorganisms (bacteria and viruses) and polyelectrolyte, and may be loaded with a light or color absorbing dye, an isotope, a radioactive species, a tag, or be porous having gas-filled pores. As used herein, the tern “hydrogel” refers to a solution of polymers, sometimes referred to as a sol, converted into gel state by small ions or polymers of the opposite charge or by chemical crosslinking.

Suitable polymers of the present invention include copolymers of water soluble polymers, including, but not limited to, dextran, derivatives of poly-methacrylamide, PEG, maleic acid, malic acid, and maleic acid anhydride and may include these polymers and a suitable coupling agent, including 1-ethyl-3(3-dimethylaminopropyl)-carbodiimide, also referred to as carbodiimide. Polymers may be degradable or nondegradable or of a polyelectrolyte material. Degradable polymer materials include poly-L-glycolic acid (PLGA), poly-DL-glycolic, poly-L-lactic acid (PLLA), PLLA-PLGA copolymers, poly(DL-lactide)-block-methoxy polyethylene glycol, polycaprolacton, poly(caprolacton)-block-methoxy polyethylene glycol (PCL-MePeg), poly(DL-lactide-co-caprolactone)-block-methoxy polyethylene glycol (PDLLACL-MePEG), some polysaccharide (e.g., hyaluronic acid, polyglycan, chitoson), proteins (e.g., fibrinogen, albumin, collagen, extracellular matrix), peptides (e.g., RGD, polyhistidine), nucleic acids (e.g., RNA, DNA, single or double stranded), viruses, bacteria, cells and cell fragments, organic or carbon-containing materials, as examples. Nondegradable materials include natural or synthetic polymeric materials (e.g., polystyrene, polypropylene, polyethylene teraphthalate, polyether urethane, polyvinyl chloride, silica, polydimethyl siloxane, acrylates, arcylamides, poly (vinylpyridine), polyacroleine, polyglutaraldehyde), some polysaccharides (e.g., hydroxypropyl cellulose, cellulose derivatives, dextran®, dextrose, sucrose, ficoll®, percoll®, arabinogalactan, starch), and hydrogels (e.g., polyethylene glycol, ethylene vinyl acetate, N-isopropylacrylamide, polyamine, polyethyleneimine, poly-aluminuin chloride).

Should the nanoparticles of the present invention require an additional layer or coating, typical suitable layers include, as examples, surfactants such as those including fatty acid esters of glycerols, sorbitol and other multifunctional alcohols (e.g., glycerol monostearate, sorbitan monolaurate, sorbitan monoleate), polysorbates, poloxamers, poloxamines, polyoxyethylene ethers and polyoxyethylene esters, ethoxylated triglycerides, ethoxylated phenols and ethoxylated diphenols, surfactants of the Genapol TM and Bauki series, metal salts of fatty acids, metal salts of fatty alcohol sulfates, sodium lauryl sulfate, and metal salts of sulfosuccinates.

The particles of the present invention are produced by conventional methods known to those of ordinary skill in the art. Techniques include emulsion polymerization in a continuous aqueous phase, emulsion polymerization in continuous organic phase, interfacial polymerization, solvent deposition, solvent evaporation, dissolvation of an organic polymer solution, cross-linking of water-soluble polymers in emulsion, dissolvation of macromolecules, and carbohydrate cross-linking. These fabrication methods can be performed with a wide range of polymer materials as described above. Removal of any solvent or emulsifier as required may include a number of methods well known to one of ordinary skill in the art. Examples of materials and fabrication methods for making nanoparticles have been published. (See Kreuter, J. 1991, Nanoparticles-preparation and applications; In: M. Donbrow (Ed.), Microcapsules and nanoparticles in medicine and pharmacy. CRC Press, Boca Raton, Fla., pp. 125-148; Hu, Z, Gao J. Optical properties of N-isopropylacrylamide microgel spheres in water. Langmuir 2002;18:1306-67; Ghezzo E, et al., Hyaluronic acid derivative microspheres as NGF delivery devices: Preparation methods and in vitro release characterization. Int J Pharm 1992;87:21-29; all references incorporated herein by reference).

Nanocoatings may be made to specifically accumulate certain cells, proteins, growth factors, peptides, biological substances and chemicals. In these cases, nanoparticles may be “tagged” to have a high affinity to specific biological component(s). In fact, a coating made of such cell/protein-affinity particles or “tags” may increase the specific accumulation of cells and proteins. When a “tag” is in contact with a nanoparticle of the present invention, it may be adsorbed or absorbed to a premade nanoparticle, or incorporated into the nanoparticle during the manufacturing process. Methods of absorption, adsorption, and incorporation are of common knowledge to those skilled in the art. The choice of the monomer and/or polymer, the solvent, the emulsifier, the tag and other auxiliary substances used herein will be dictated by the nanoparticle being fabricated and is chosen, without limitation and difficulty, by those skilled in the art. The ratio of tag to nanoparticle may be varied as required.

As used herein, a “tag” includes an addition to the nanoparticle that has an ability to modify the nanoparticle. Such tags may include drugs, molecular ligands (e.g., molecules/compounds) that recognize a material, cell, organ or tissue of interest, such as antibodies, antigens, proteins, peptides, nucleic acid sequences, fatty acid or carbohydrate moieties, chemicals, as examples. They may also be modified compounds or polymers that mimic recognition sites on cells, organs, or tissues. The tags may recognize a portion of a material, cell, organ, or tissue, including but not limited to a cell surface marker, cell surface receptor, immune complex, antibody, MHC, extracellular matrix protein, plasma, cell membrane, extracellular protein, polypeptide, cofactor, growth factor, fatty acid, lipid, carbohydrate chain, gene sequence, cytokine or other polymer.

Nanoparticles of the present invention may be applied to the surface of an implant by methods known to one of ordinary skill in the art, including by physical adsorption or chemical conjugation. The techniques described in accordance with the present invention may be used in vivo and in vitro. For example, nanoparticles can be used for coating blood bags and/or blood tubes. Techniques for making particles and coating implants in accordance with the present invention are further described by examples presented below.

Examples of Nanoparticle Preparation and Biocompatibility

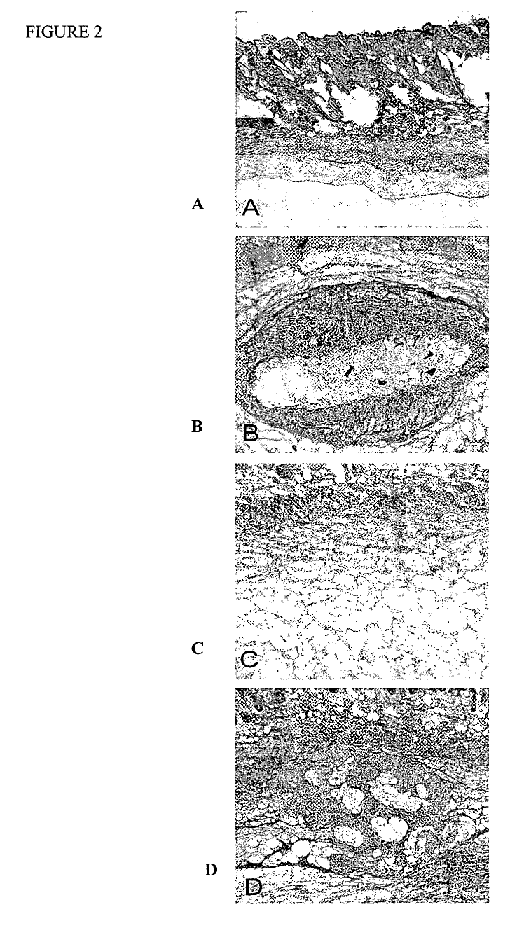

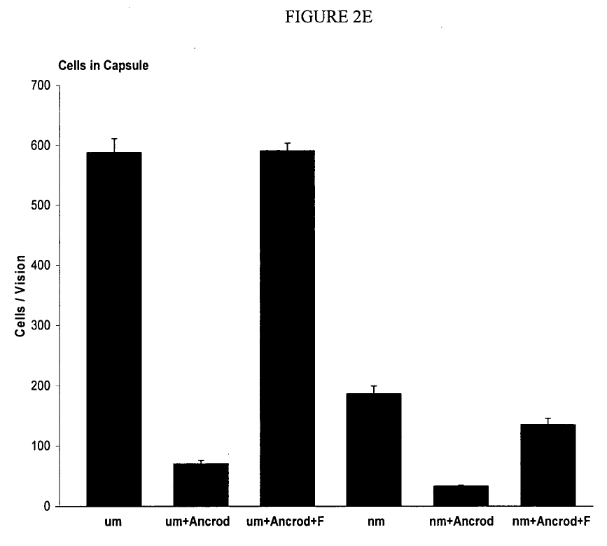

N-isopropylacrylamide (NIPA) particles and hydro-propyl cellulose (HPC) particles were produced in sizes ranging from 100 nm to 20 μm. The particles were implanted in a subcutaneous space of Balb/C mice. After implantation for periods ranging from 3 days to 21 days, it was determined that adverse and foreign body reactions, such as inflammatory and fibrotic responses, were absent or less evident when smaller particles were implanted. Such size-dependence related to adverse tissue responses was independent of the material (i.e., particle) composition. In general, particles with sizes less than 500 nm showed the least adverse responses as shown in FIG. 2A and B.

FIGS. 2, 2A and 2B are photos taken at 200× and show the absence or presence of adverse or foreign body reactions to NIPA nanoparticles of the present invention seven days after implantation in the subcutaneous space of Balb/C mice. In FIG. 2A, NIPA particles 100 nanometers in diameter were found to illicit minimal foreign body reactions (e.g., inflammation) as compared with NIPA particles that were 10 micrometers in diameter, as shown in FIG. 2B.

Fibronogen-depleted mice, also referred to a hypofibrinogenemic mice, were generated by repeat administering ancrod (a snake venom) to the mice 3 days prior to implantation. These hypofibrinogenemic mice failed to illicit adverse or foreign body reactions to particles that were 10 micrometers in diameter, as shown in FIG. 2C, because of the depletion of fibrinogen. When these same particles were preincubated with fibrinogen (supplemented with fibrinogen) at 3 microgram/mL for 4 hours before implantation in hypofibrinogenemic mice, the adverse responses were again observed. Thus, when fibrinogen was able to adsorb to the larger particles, an adverse response (such as inflammation) would occur even in hypofibrinogenemic mice. The quantitative results of tissue responses to such particles of micrometer (μm) versus nanometer (nm) size is summarized in FIG. 2E.

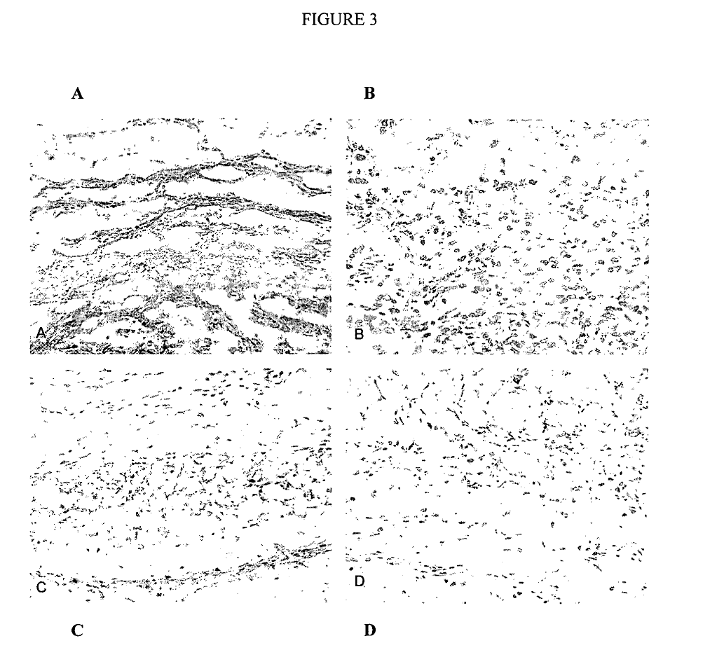

Previous work by the inventor has shown that denatured fibrinogen will bind to a biomaterial or particle of larger dimensions and results in proinflammatory processes. As such, particles of larger size (e.g., 10 micrometer in diameter) were implanted subcutaneously in Balb/c mice using a subcutaneous implant model. Large amounts of fibrinogen (detected with peroxidase-conjugated antibody against fibrinogen) were found to accumulate around these larger particles as shown in FIG. 3A. Using the same mouse model with the same size particles but initially treating the mice with ancrod resulted in a greatly reduced amount of fibrinogen that accumulated around the particle implant (see FIG. 3B). These results were compared to those observed in mice in which a nanoparticle implant (diameter of about 100 nm) was implanted (FIG. 3C) and those pretreated with ancrod after which nanoparticles were implanted (FIG. 3D). In mice receiving the nanoparticle implants, very little fibrinogen denaturation or accumulation around the implantation site was observed (FIG. 3C and 3D). Fibrinogen accumulation was determined using immunohistochemical staining against mouse fibrinogen and observing tissue samples under a microscope set at 400×.

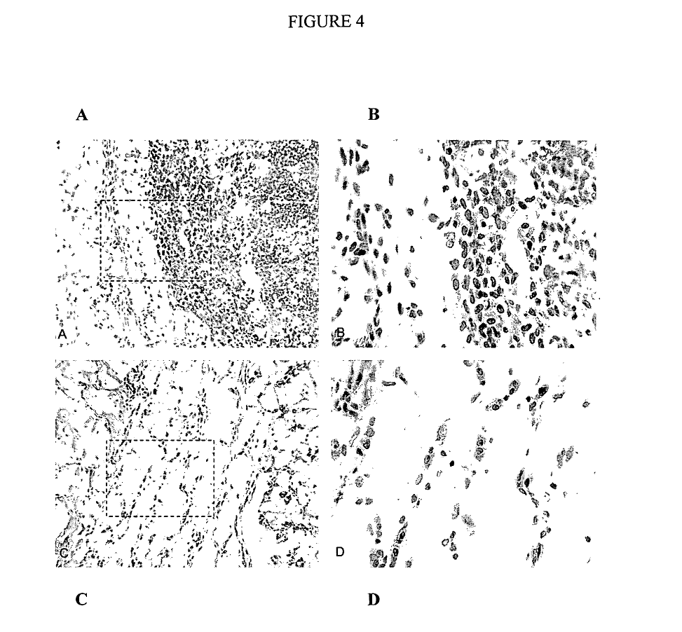

Because adverse biologic responses following insertion of an implant in an organism also include the accumulation of inflammatory cells and the formation of fibrotic capsules, these reactions were observed following implantation of larger particles and nanoparticles. As shown in FIG. 4A (100×), larger particle implants of 10 micrometer diameter were found to illicit the recruitment of CD11b-positive inflammatory cells in mice using the subcutaneous implant model. Pretreating these mice with ancrod reduced both fibrinogen accumulation (possibly denaturation) and inflammatory cell aggregation around the implantation site, as shown in FIG. 4B (100×). On the other hand, using the same implant model but implanting nanoparticles of 100 nm diameter resulted in minimal inflammatory cell accumulation around the implant site, as shown in mice in which fibrinogen levels were depleted by pretreatment with ancrod (FIG. 4D) or in which fibrinogen levels were not affected (FIG. 4C). FIG. 4C and 4D are enlarged views (400×) of the dashed boxes FIGS. 4A and 4B, respectively. The extent of the inflammatory response to particle implants was assessed using immunohistochemical staining against CD11b-positive inflammatory cells.

Examples of Coating with Nanoparticles and their Biocompatibility

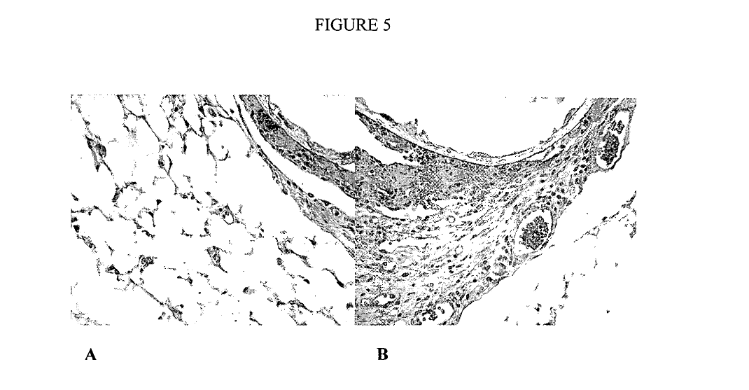

Poly-L-lactic acid (PLLA) fibers were coated with nanoparticles of 100 nm diameter. NIPA nanoparticle-coated fibers were introduced into mice using the subcutaneous implantation mode and tissue samples were then examined seven days after implantation. FIG. 5A shows that fibers coated with such nanoparticles did not produce adverse biologic responses such as inflammation and inflammatory cell accumulation or protein adhesion. This was contrasted to fibers that were not coated or that were coated with larger particles (micrometer in diameter). With uncoated or larger-coated fibers, adverse responses and foreign body reactions were elicited (FIG. 5B).

Similarly, adverse reactions were not apparent when implanting PET films coated with 100 nm diameter nanoparticles using the subcutaneous implant model, while reactions were apparent when implanting PET films coated with larger particles (micrometer in diameter). (Data not shown). Here, coating with nanoparticles, with diameters less than 500 nm, significantly reduced the accumulation of phagocytic cells by greater than 70% and reduced fibrotic tissue formation by greater than 50%. Similar studies using hydroxl propyl cellulose (HPC) particles as coating material yield similar results.

Nanoparticles can be physically or chemically conjugated to a large variety of materials, including nondegradable polymers, degradable polymers, metal, hydrogel, carbon, proteins, organic/inorganic chemicals, drugs, biological polymers, phospholipid polymers, dental materials, bone materials and soft tissue materials.

Example Nanoparticles Preventing Protein Denaturation

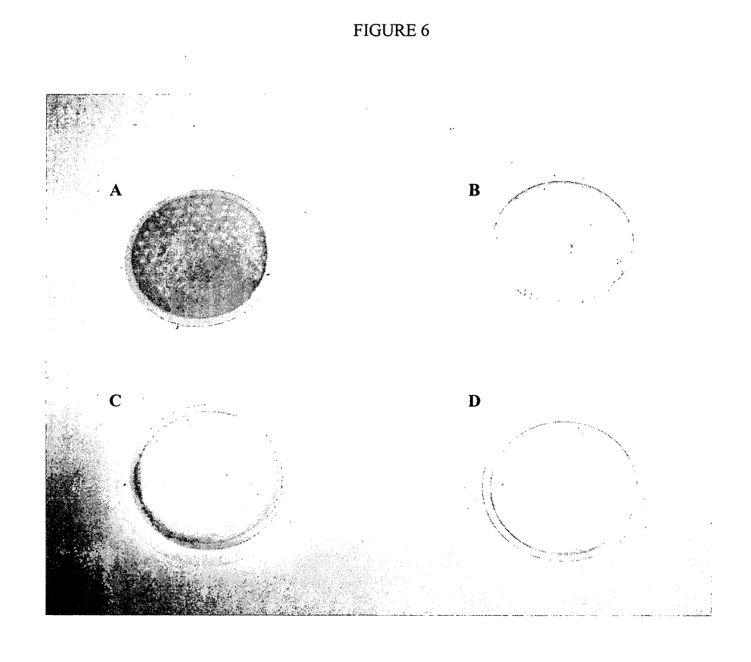

Using an in vitro model, it has been found that larger particles (e.g., those micrometer in diameter) are capable of denaturing fibrinogen (FIG. 6A), while smaller, nanoparticles (of at least about 100 nm in diameter or less than 500 nm) prevent protein denaturation (FIG. 6C). The extent of particle-mediated fibrinogen denaturation was assessed using an enzyme-linked immunoabsorption assay (ELISA) and the fibrinogen P2 epitope. Here, both larger particles of 10 micrometer diameter and nanoparticles of about 100 nanometer diameter were incubated with human fibrinogen at 1 mg/mL for 4 hours at 37 degrees Centigrade. Then NIPA particles were then subjected to the ELISA assay with the P2 epitope following standard procedures. FIG. 6A demonstrated that there was an increase in P2 exposure with larger particles (A) trigger much more P2 exposure than did nanoparticles (C). The fibrinogen-free microparticles (C) and nanoparticles (D) have very low affinity to P2 antibody. Similar results have also been obtained from studies using HPC particles (not shown).

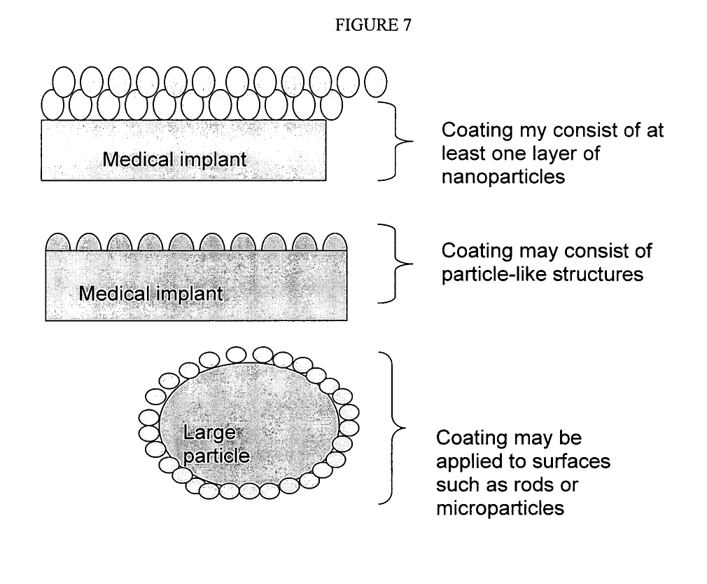

Nanoparticles of the present invention provide for a coating on an implant surface to be implanted into an organism in need thereof. The coating may be applied to any material via physical and/or chemical binding, including techniques such as plasma polymerization or spot coating. In general, the coating of the present invention when applied to an implant surface is used for purposes that may be cosmetic, therapeutic, preventative, reconstructive, monitoring and replacement. In addition, the coating of the present invention may be used for in vitro purposes. FIG. 7 illustrates that such a coating is generally at least one layer thick, may include particle-like structures (e.g., using plasma polymerization, spot coating, laser deposition, and related technologies) and may also be used on implant surfaces such as small 2 mm rods or microparticles.

Additional objects, advantages and novel features of the invention as set forth in the description, will be apparent to one skilled in the art after reading the foregoing detailed description or may be learned by practice of the invention. The objects and advantages of the invention may be realized and attained by means of the instruments and combinations particularly pointed out here.

1. A nanoparticle preparation for coating an implant surface comprising:
one or more nanoparticles of less than or equal to 500 nanometers; and
an implant surface capable of receiving the nanoparticles.

2. The nanoparticle preparation of claim 1, wherein the nanoparticles are selected from the group consisting of metal, carbon, graphite, polymer, hydrogel, protein, peptide, nucleic acid, bacteria, virus, liquid, porous and polymer blend particles and combinations thereof.

3. The nanoparticle preparation of claim 1, wherein the nanoparticles promote characteristics on the implant surface after implementation into an organism in need thereof, the characteristics selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof.

4. The nanoparticle preparation of claim 1 further comprising a surfactant on the surface of the nanoparticle.

5. The nanoparticle preparation of claim 4, wherein the surfactant is selected from the group consisting of fatty acid esters of glycerols, sorbitol, multifunctional alcohols, glycerol monostearate, sorbitan monolaurate, sorbitan monoleate, polysorbates, poloaxmers, poloaximines, polyoxyethylene ethers and polyoxyethylene esters, ethosylated tryglycerides, ethoxylated phenols and ethoxylated diphenols, surfactants of the Genapol TM and Bauki series, metal salts of fatty acids, metal salts of fatty alcohol sulfates, sodium lauryl sulfate, metal salts of sulfosuccinates and combinations thereof.

6. The nanoparticle preparation of claim 1 further comprising a tag in contact with the nanoparticle, wherein contact is selected from the group consisting of adsorption, absorption, incorporation and combinations thereof.

7. The nanoparticle preparation of claim 6, wherein the tag recognizes materials selected from the group consisting of a cell, protein, peptide, DNA, RNA, micro-organism, virus, bacteria, molecular ligand, organ, tissue and combinations thereof.

8. The nanoparticle preparation of claim 6, wherein the tag is selected from the group consisting of drugs, molecular ligands, antibodies, antigens, proteins, peptides, nucleic acid sequences, fatty acids, carbohydrate moieties, chemicals and combinations thereof.

9. The nanoparticle preparation of claim 1, wherein the implant has uses selected from the group consisting of cosmetic, therapeutic, preventative, reconstructive, for monitoring, and combinations thereof.

10. The nanoparticle preparation of claim 1, wherein the nanoparticles are selected from the group consisting of N-isopropylacrylamide, hydro-propyl cellulose, poly-L-lactic acid and combinations thereof.

11. A nanoparticle preparation for coating an implant surface comprising:
nanoparticles of less than or equal to 500 nanometers, wherein the nanoparticles promote characteristics on the implant surface after implantation into an organism in need thereof, the characteristics selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof.

12. The nanoparticle preparation of claim 11, wherein the nanoparticles are selected from the group consisting of metal, carbon, graphite, polymer, hydrogel, liquid, protein, peptide, nucleic acids, microorganisms, bacteria, viruses, porous and polymer blend particles and combinations thereof.

13. The nanoparticle preparation of claim 11 further comprising a surfactant on the surface of the nanoparticle.

14. The nanoparticle preparation of claim 13, wherein the surfactant is selected from the group consisting of fatty acid esters of glycerols, sorbitol, multifunctional alcohols, glycerol monostearate, sorbitan monolaurate, sorbitan monoleate, polysorbates, poloaxmers, poloaximines, polyoxyethylene ethers and polyoxyethylene esters, ethosylated tryglycerides, ethoxylated phenols and ethoxylated diphenols, surfactants of the Genapol TM and Bauki series, metal salts of fatty acids, metal salts of fatty alcohol sulfates, sodium lauryl sulfate, metal salts of sulfosuccinates and combinations thereof.

15. The nanoparticle preparation of claim 11 further comprising a tag in contact with the nanoparticle, wherein contact is selected from the group consisting of adsorption, absorption, incorporation, and combinations thereof.

16. The nanoparticle preparation of claim 15, wherein the tag recognizes materials selected from the group consisting of a cell, micro-organism, protein, molecular ligand, organ, tissue and combinations thereof.

17. The nanoparticle preparation of claim 15, wherein the tag is selected from the group consisting of drugs, molecular ligands, antibodies, antigens, proteins, peptides, nucleic acid sequences, fatty acids, carbohydrate moieties, chemicals and combinations thereof.

18. The nanoparticle preparation of claim 11, wherein the implant has uses selected from the group consisting of cosmetic, therapeutic, preventative, replacement, reconstructive, for monitoring, and combinations thereof.

19. The nanoparticle preparation of claim 11, wherein the nanoparticles are selected from the group consisting of N-isopropylacrylamide, hydro-propyl cellulose, poly-L-lactic acid and combinations thereof.

20. A method of preparing nanoparticles for coating an implant surface comprising the steps of:
selecting nanoparticles of less than or equal to 500 nanometers; and
coating the surface of an implant with nanoparticles,
wherein nanoparticles promote characteristics on the implant surface selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof.

21. The method of claim 20 further comprising the step of selecting nanoparticles from the group consisting of metal, carbon, graphite, polymer, hydrogel, liquid, porous or polymer blend particles and combination thereof.

22. The method of claim 20 further comprising the step of adding a surfactant to the surface of the nanoparticle.

23. The method of claim 22, wherein the surfactant is selected from the group consisting of fatty acid esters of glycerols, sorbitol, multifunctional alcohols, glycerol monostearate, sorbitan monolaurate, sorbitan monoleate, polysorbates, poloaxmers, poloaximines, polyoxyethylene ethers and polyoxyethylene esters, ethosylated tryglycerides, ethoxylated phenols and ethoxylated diphenols, surfactants of the Genapol TM and Bauki series, metal salts of fatty acids, metal salts of fatty alcohol sulfates, sodium lauryl sulfate, metal salts of sulfosuccinates and combinations thereof.

24. The method of claim 20 further comprising the step of including a tag in contact with the nanoparticles, wherein contact is selected from the group consisting of adsorption, absorption, incorporation and combinations thereof.

25. The method of claim 24, wherein the tag recognizes a material selected from the group consisting of a cell, protein, nucleic acid, microorganism, bacteria, virus, peptide, molecular ligand, organ, tissue and combinations thereof.

26. The method of claim 24, wherein the tag is selected from the group consisting of drugs, molecular ligands, antibodies, antigens, proteins, peptides, nucleic acid sequences, fatty acids, carbohydrate moieties, chemicals and combinations thereof.

27. The method of claim 20, wherein the implant has uses selected from the group consisting of cosmetic, therapeutic, preventative, replacement, reconstructive, for monitoring, and combinations thereof.

28. The method of claim 20, wherein the nanoparticles are selected from the group consisting of N-isopropylacrylamide, hydro-propyl cellulose, poly-L-lactic acid and combinations thereof.

29. A nanoparticle preparation for coating an implant surface comprising:
one or more nanoparticles of less than or equal to 500 nanometers; and
an implant surface containing poly-L-lactic acid fibers capable of receiving the nanoparticles,
wherein the nanoparticles promote characteristics on the implant surface after implantation into an organism in need thereof the characteristics selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof.

30. The nanoparticle preparation of claim 29 further comprising a surfactant on the surface of the nanoparticle.

31. The nanoparticle preparation of claim 30, wherein the surfactant is selected from the group consisting of fatty acid esters of glycerols, sorbitol, multifunctional alcohols, glycerol monostearate, sorbitan monolaurate, sorbitan monoleate, polysorbates, poloaxmers, poloaximines, polyoxyethylene ethers and polyoxyethylene esters, ethosylated tryglycerides, ethoxylated phenols and ethoxylated diphenols, surfactants of the Genapol TM and Bauki series, metal salts of fatty acids, metal salts of fatty alcohol sulfates, sodium lauryl sulfate, metal salts of sulfosuccinates and combinations thereof.

32. The nanoparticle preparation of claim 29 further comprising a tag in contact with the nanoparticle, wherein contact is selected from the group consisting of adsorption, absorption, incorporation and combinations thereof.

33. The nanoparticle preparation of claim 32, wherein the tag recognizes materials selected from the group consisting of a cell, protein, molecular ligand, organ, tissue and combinations thereof.

34. The nanoparticle preparation of claim 32, wherein the tag is selected from the group consisting of drugs, molecular ligands, antibodies, antigens, proteins, peptides, nucleic acid sequences, fatty acids, carbohydrate moieties, chemicals and combinations thereof.

35. A nanoparticle preparation for coating an implant surface comprising:
one or more nanoparticles of less than or equal to 500 nanometers; and
an implant surface containing a PET film capable of receiving the nanoparticles,
wherein nanoparticles promote characteristics on the implant surface selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof.

36. The nanoparticle preparation of claim 35 further comprising a surfactant on the surface of the nanoparticle.

37. The nanoparticle preparation of claim 36, wherein the surfactant is selected from the group consisting of fatty acid esters of glycerols, sorbitol, multifunctional alcohols, glycerol monostearate, sorbitan monolaurate, sorbitan monoleate, polysorbates, poloaxmers, poloaximines, polyoxyethylene ethers and polyoxyethylene esters, ethosylated tryglycerides, ethoxylated phenols and ethoxylated diphenols, surfactants of the Genapol TM and Bauki series, metal salts of fatty acids, metal salts of fatty alcohol sulfates, sodium lauryl sulfate, metal salts of sulfosuccinates and combinations thereof.

38. The nanoparticle preparation of claim 35 further comprising a tag in contact with the nanoparticle, wherein contact is selected from the group consisting of adsorption, absorption and incorporation and combinations thereof.

39. The nanoparticle preparation of claim 38, wherein the tag recognizes materials selected from the group consisting of a cell, protein, DNA, RNA, peptide, microorganisms, bacteria, viruses, molecular ligand, organ, tissue and combinations thereof.

40. The nanoparticle preparation of claim 38, wherein the tag is selected from the group consisting of drugs, molecular ligands, antibodies, antigens, proteins, peptides, nucleic acid sequences, fatty acids, carbohydrate moieties, chemicals and combinations thereof.

41. The nanoparticle preparation of claim 35, wherein the implant surface is selected from the group consisting of nondegradable polymers, degradable polymers, metal, hydrogel, carbon, proteins, organic chemicals, inorganic chemicals, drugs, biological polymers, phospholipids polymer, dental materials, bone materials, soft tissue materials and combinations thereof.

42. A nanoparticle preparation for implant surfaces comprising:
one or more nanoparticles of less than or equal to 500 nanometers, wherein the nanoparticles promote characteristics selected from the group consisting of reducing protein unfolding, reducing protein denaturation, preventing accumulation of inflammatory cells, preventing the accumulation of fibrotic cells, preventing fibrotic tissue formation, preventing thrombosis or device-centered infection, reducing the number of cell attachment sites, reducing adverse biological reactions and combinations thereof; and
an implant surface capable of receiving the nanoparticles.

43. The nanoparticle preparation of claim 42, wherein the implant surface is modified by a surface modification procedure selected from the group consisting of plasma polymerization, spot coating and combinations thereof.

44. The nanoparticle preparation of claim 43, wherein modifying the implant surface creates nanoparticles on the surface.
